(19) United States
(12) Patent Application Publication
(10) Pub. No.: US 2018/0085276 A1
(43) Pub. Date: Mar. 29, 2018

(54) DEVICE FOR DRIVING THE LOWER LIMBS OF A PERSON IN DORSAL OR PARTIAL DECUBITUS COMBINED WITH DRIVING WALKING IN VERTICAL POSITION

(71) Applicant: RB Patents SARL, Villeneuve (CH)

(72) Inventor: Roland Brodard, Villeneuve (CH)

(21) Appl. No.: 15/563,157

(22) PCT Filed: Mar. 24, 2016
(86) PCT No.: PCT/IB2016/051673

§ 371 Date: Sep. 29, 2017

(30) Foreign Application Priority Data
Apr. 1, 2015 (CH) 00470/15

(51) Int. Cl. A61H 1/02; A61N 1/04; A61N 1/36

(52) U.S. Cl. CPC A61H 1/0262; A61H 1/0266; A61N 1/0452; A61N 1/36003; A61N 1/0484; A61H 2201/10; A61H 2230/085; A61H 2201/1238; A61H 2201/5007; A61H 2201/5061; A61H 2201/5064; A61H 2201/1642; A61H 2203/0456; A61H 2201/1652

(57) ABSTRACT

The invention relates to a device for driving the lower limbs of a person, including a base frame; a table supporting the person; at least one motorized mechanical orthosis arranged to constitute an interface with at least one of the lower limbs of the person so that the movements of the lower limb and the orthosis are connected and identical and with the orthosis being attached to one end of the table; and a device for functional electrical stimulation and for measuring an electromyogram including at least one pair of stimulation and measurement electrodes intended for acting on a muscle or muscle group of the lower limb, and for stimulating the muscle or muscle group, as well as for measuring the reaction of the muscle or muscle group, wherein the device also includes a raising mechanism which makes it possible to vary the vertical position of the table relative to the base frame between a low position in which the transfer and the installation of the person are made easier, intermediate working positions, and a raised position making it possible to drive the person in a standing position, and a mechanism for tilting the table which makes it possible to vary the inclination of the table relative to the base frame, in particular between a horizontal position in which the person is positioned in dorsal decubitus, and a vertical position in which the person is in a standing position, and wherein the combination of the mechanisms for raising and tilting the table allowing the mobility of the orthosis across the entire respective physiological ranges of movement of the lower limb.

### FIELD OF THE INVENTION

The field of the invention relates to a device allowing for the re-education and/or the driving of the mobility, the force and the endurance of the lower limbs of a person in dorsal or partial decubitus position combined with the driving of the gait in vertical position, particularly for rehabilitation following a motor or neuromotor handicap, such as, for example, paraplegia, hemiplegia or, in case of cerebral palsy; and in the sport domain for post-traumatic functional re-education and/or driving.

### STATE OF THE ART

Devices exercising the mobility of the lower limbs of a person in recumbent or seated position are known; they are either simple pedaling devices, generally of ergometric bicycle type, such as, for example, the StimMaster and ERGYS2 devices, or devices allowing for the extension of the legs against a load resistance, usually called “leg press”, such as, for example, the CON-TREX device. Some devices benefit from the assistance of electrical neuromuscular stimulation.

The most advanced of these devices for mobilizing the lower limbs with exoskeleton-type structure, assisted by an electrical neuromuscular stimulation set in closed loop mode, is described in the patent U.S. Pat. No. 7,381,192 and is marketed under the marketing reference Motionmaker™. This device makes it possible to drive the mobility, the muscle force and the endurance over the entire extent of the physiological range of the articulations of the lower limbs. This device does however present a few drawbacks. In particular, it is not capable of rapid movements because the electric motors actuating the exoskeleton-type structure have limited power because their dimensions are also limited because they are fixed to the mobile exoskeleton. For the same reasons, the maximum force delivered by this device in a lower limb extension exercise of the “leg press” type is only 250 Newtons per leg, very inadequate to allow the driving of healthy people and/or people with only paresis, the force necessary for the “leg press” type driving of a healthy person having to be of the order of 2000 Newtons per leg.

Other devices exercising the driving of the gait of a person in standing position are known; they are more often than not devices utilizing a motorized treadmill on which the user is supported by a system relieving his or her weight usually by means of a harness of parachutist type and whose gait on the treadmill is either assisted by a leg orthosis or by a leg brace, whereas the weight of these elements can be supported by complementary relieving elements such as, for example, a parallelogram-shaped support. The patent U.S. Pat. No. 6,821,233 describes such a device. A variant of this type of device replaces the motorized treadmill with a system composed of two motorized moving plates, each of these plates being intended to support a foot of the user. Said plates perform an alternating forward and backward motorized movement which determines an alternate movement of the legs which is like the gait movement. The GangTrainer and HapticWalker devices use this variant. Another device described by the patent U.S. Pat. No. 6,685,658 consists of a motorized table for arranging the user vertically, enabling him or her to switch from the dorsal decubitus position to the vertical position with the feet then resting on two movable plates with alternate motorized movement.

The devices described above in the context of the prior art have the drawback of not being multipurpose because they are either specific devices which exercise, in recumbent or seated position, the mobility of the lower limbs and the driving of the force and of the endurance of said mobility, or of other specific devices which exercise, in vertical position, a driving of the gait.

It is therefore necessary to improve the proposed systems and methods, to better address the physiological demands of total and early rehabilitation of the motrice and locomotive function of the lower limbs, and to facilitate and simplify the task of the system operator, while guaranteeing a totally reliable and effective solution.

The aim of the invention is to be able to perform, by means of a single and multipurpose device, an early total functional re-education of the lower limbs of a neurological patient (paralyzed or paretic) and/or the driving of a healthy person by combining, in dorsal or partial decubitus position, the driving of the mobility, of the force and of the endurance of said limbs against an adjustable load resistance with, in standing (vertical) position, the early driving of the gait.

### DESCRIPTION OF THE INVENTION

The present invention relates to a device for driving the lower limbs of a person in dorsal or partial decubitus position combined with the driving of the gait in vertical position.

It is well known that the result of the immobilization of the limbs, in particular of a paralysis, is a heavy functional handicap which can be aggravated by a whole series of complications: muscular atrophy, eschars, spasticity, osteoporosis, circulatory disorders or muscular-tendinous as well as capsular-ligamentary retractions. A muscular-tendinous retraction has an associative muscular atrophy. The result thereof is that the muscle loses its force and its endurance. Consequently, it loses its capacity to supply functional work.

The capsular-ligamentary retraction is also a major and very common complication which can provoke a limitation of articular amplitude (ankyloses) and abnormal postures. With time, it can also have a repercussion on the articular cartilage.

It is therefore essential to prevent these various complications, and in particular the musculo-tendino-articular problems, by regularly mobilizing the patient from the start of his or her injury by a well-established program based on the injury, to strengthen the musculature and the endurance of the lower limbs.

It is also essential for, as rapidly as possible, the patient to be placed in vertical position (standing position), in order for him or her to be in a physiological position ensuring a regularization of the different metabolisms and of numerous physiological functions, then to begin an early driving of the locomotive function which is one of the main objectives in order to restore the gait to an optimal level.

Ideally, the drivings of mobility of the limbs and of the gait must be started at the earliest possible time compatible with the specific case of a given patient. Globally, the driving must make it possible to avoid having the inactivity further promote the neglect of the motor function and, in neurological patients, the neural degeneration with loss of motor function. The driving is important to retain neural activity until a potential regeneration of the voluntary actions is revealed.

In neurological patients, the neural impairment is most often partial, and for example only 20% of paraplegic people have suffered a total impairment of the spinal cord, whereas 80% show only a partial impairment of the spinal cord.

Most neurological patients, having a partial cerebral or medullary neural impairment exhibit a functional recovery potential based on the great “plasticity” faculty of the central nervous system and in particular on its “plasticity of substitution” capacity, that is to say that, as a result of a reorganization of the sub-lesional nerve circuits, unimpaired, still healthy, nerve circuits, can be substituted for destroyed nerve circuits to ensure the function thereof.

This plasticity of substitution can be stimulated because, for the most part, it is determined by the nerve signals supplied to the central nervous system by the proprioceptive nervous system and, even more specifically, by the closed adjustment loop produced between the proprioceptive nervous system and the motor nerves (alpha motor neurons) of the muscles concerned.

It is worth recalling here an essential point: the contraction of any muscle responsible for a movement is under the control of the proprioceptive nervous system on which said muscle depends. This nervous system comprises proprioceptors, which are receptors, at the origin of a sensitive nervous fiber, sensitive to the stimulations produced by the movements of the body. These receptors are situated in the vicinity of the bones, of the articulations and of the muscles. The proprioceptive nervous system (which represents the deep sensitivity) forms, with the muscles that it controls, a closed loop adjustment system, subtle and accurate, which allows the control of the movements and of the position of the body.

In the context of the proprioceptive nervous system, the muscles play a major role because they contain two essential proprioceptors, the neuromuscular spindles, which predominantly inform the central nervous system of the position and of the movement of the segments of the limbs of the body in relation to its environment by the transmission of information concerning the length of the muscles to which they belong and the rate of variation of this length and the Golgi tendon organs by their transmission of information concerning the degree of tension (force) of the tendon to which they belong and consequently the force exerted by the muscle concerned.

The result of the above is that these two intramuscular proprioceptors play a primordial role in the control of the position and of the movements. However, a feature common to these two types of intramuscular proprioceptors is that they are sensitive only to their stretching and that they remain silent in the absence of stretching.

Together, the information supplied by the intramuscular proprioceptors allows for a rigorous control of the contraction of the muscle, by the modulation of the motor nerve conduction, transmitted to this muscle by its motoneurons of medullary origin, in other words by its alpha motoneurons.

The following example fully demonstrates the importance of this common primordial feature. Normally, the voluntary contraction of a flexing muscle of a given articulation provokes a flexing movement of said articulation. This contraction, with shortening of said muscle, determines a tension of the tendons of the muscle whose degree of force is transmitted to the central nervous system by the Golgi tendon organs, whereas the neuromuscular spindles of said muscle are co-activated (by the gamma motoneurons) with the contraction of this muscle and transmit to the central nervous system, in real time, the length of the muscle and the rate of variation of this length. In this case, all the proprioceptive information relating to the articular flexing movement transmitted to the central nervous system is correct.

In the case where, in contrast to a voluntary movement, the flexing movement of said articulation is provoked passively by an external means, manual or robotic, the induced movement provokes the passive shortening imposed on the muscle, there is consequently no tension transmitted to the tendons of said muscle and in the absence of tension, the Golgi organs remain silent, and the neuromuscular spindles, which are not co-activated by this passive shortening of the muscle consequently also remain silent. In this case, no proprioceptive information is transmitted to the central nervous system.

The above example then underscores the major drawback resulting from a driving by the passive movements imposed by a manual or robotic external means. It becomes obvious that such movements do not transmit to the central nervous system the precious proprioceptive information essential to the effective stimulation of the plasticity of substitution and that they can even prove counterproductive.

The result of the above is that the active participation of the muscles in an effective driving of the lower limbs is imperative and cannot be ignored.

Thus, in neurological patients who exhibit a deficiency or an absence of motoneuron control of the voluntary muscular contraction, a functional electrical stimulation (FES) of the paretic or paralyzed muscles adjusted in closed loop mode in real time is the solution of choice for provoking the controlled muscular contractions, capable of transmitting the appropriate proprioceptive information to the central nervous system.

Another advantage of this stimulation of CLIMFES (Closed-Loop Integrated Myography Functional Electrical Stimulation) type is to make it possible to produce and control, in force, rate and articular amplitude, all the physiological articular movements of the limbs.

The key objective of reeducational driving is to allow the patient to recover, then sustain, a functional working capability (somewhat like a “fitness” level) of the lower limbs which, to the greatest possible extent, are capable of supporting the weight of the body in vertical position, or even better, are capable of switching from a seated position, even crouched position, to the vertical position, and vice versa. At this stage only, the patient will be genuinely capable of being able to undertake early, with optimum benefit, a specific driving of the bipedal gait process.

Achieving the objective described above preferentially involves driving motor patterns of the movements and of gait that are as real and normal as possible. For that, it is essential to ensure that the proprioceptive information, supplied by the muscles to the central nervous system, is as close as possible to the information resulting from a similar voluntary movement. The result thereof is that the driven movements must be performed by respecting the closest possible mimicry of the initial voluntary movements, with an active participation of the muscles concerned and by scrupulously respecting the load resistances which opposed these initial movements.

In the specific case of the early driving of the bipedal gait process, it is sufficient initially to scrupulously respect the kinematics and dynamics against natural load resistance of the movements of the three segments of the lower limbs, that is to say of the sequence of the flexing-extension movements of the thigh on articulation of the hip, of the leg on articulation of the knee and of the foot on articulation of the ankle, to appropriately stimulate plasticity of substitution, then, if necessary, to be able to observe the first signs of functional motor recovery of the gait. In case of probative signs of recovery, a more elaborate driving of the natural gait on the ground taking into account in particular the specific natural movements of the pelvis will then be able to be validly considered.

An additional objective is to provide the re-educator with an early index and reliable precursor of the potential capacity for recovery of a voluntary functional activity, by means of surface electromyography (EMG) of the muscles concerned, then to be able to track the development of this recovery phase throughout the consecutive drivings.

To date, there is no single and multipurpose device for driving the lower limbs of a neurological patient in dorsal or partial decubitus position combined with the driving of the gait in vertical position, which fulfils the conditions stated above, so as to be able to drive the lower limbs physiologically, respecting a close mimicry of the voluntary activity that has become impossible or restricted, following an injury to the central nervous system and for which it is possible to assess early signs of a functional motor recovery and its degree of evolution in time by means of an integrated surface electromyography system (EMG).

The aim of the present invention is to propose a single and multipurpose device for driving the lower limbs combined with the early driving in gait which is free of the drawbacks (defects) listed above and which satisfies the conditions stated above, so as to ensure an optimal physiological driving to reeducate the lower limbs, then driving in gait, by respecting the closest possible mimicry of a voluntary active driving and incorporating the assessment of the results and their evolution over time by means of an integrated surface electromyography (EMG).

The functional combination in a single multipurpose device of all of the functionalities, usually assigned to two distinct types of devices, offers the advantage that such a device, single and compact, makes it possible to save on a lot of space and greatly facilitate the work of the operator, in particular through the extent of the functionalities available on the same device.

To this end, the invention relates to a device for driving the lower limbs as defined in claim 1.

### DETAILED DESCRIPTION

The following description, given as an exemplary embodiment, refers to the drawings in which:

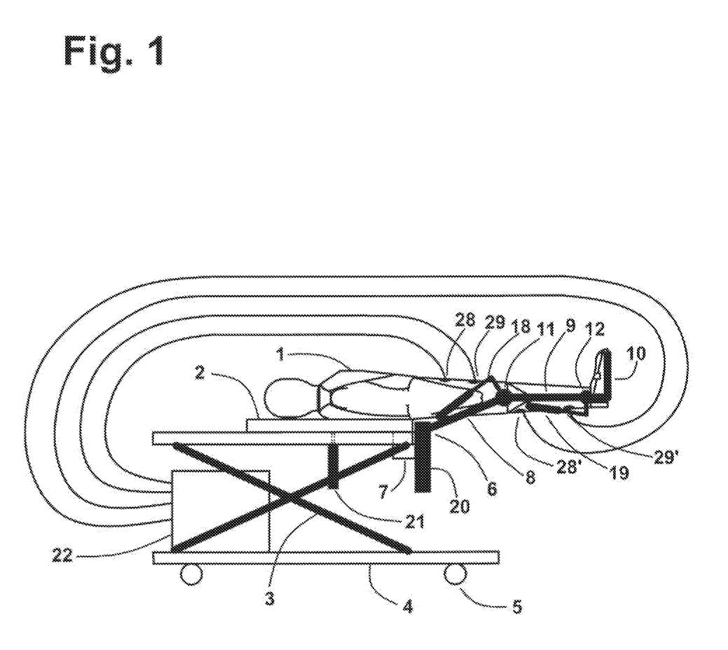

FIG. 1 schematically illustrates a mode of execution of the device according to the invention in its low horizontal position allowing the initial installation of the patient in dorsal decubitus position;

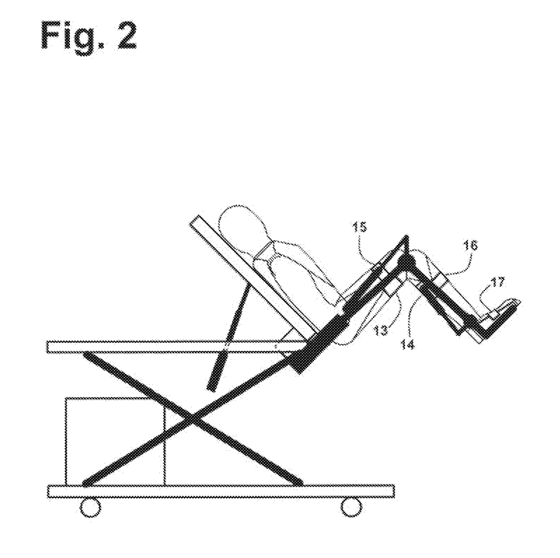

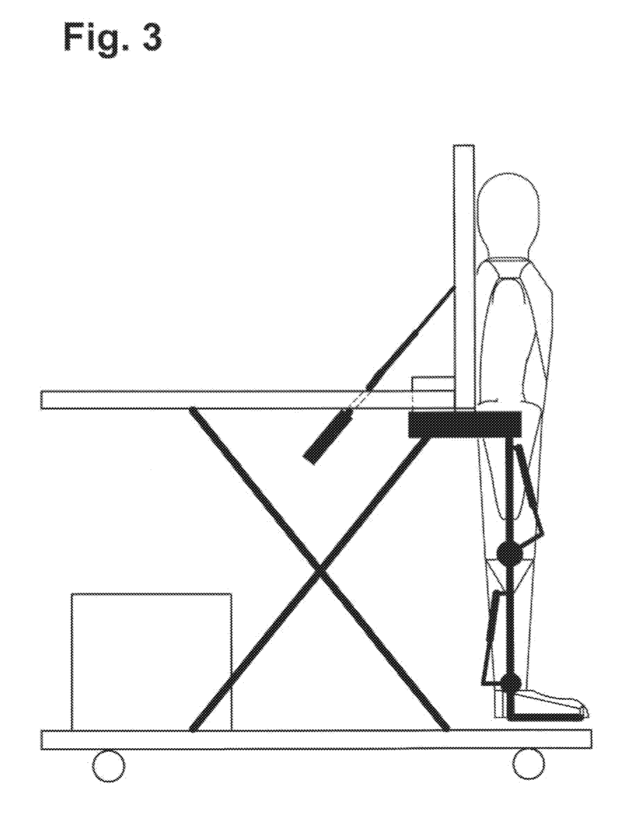

FIGS. 2 and 3 schematically show two positions, respectively an intermediate gradual position in partial decubitus position in FIG. 2 and the vertical position (patient standing) in FIG. 3;

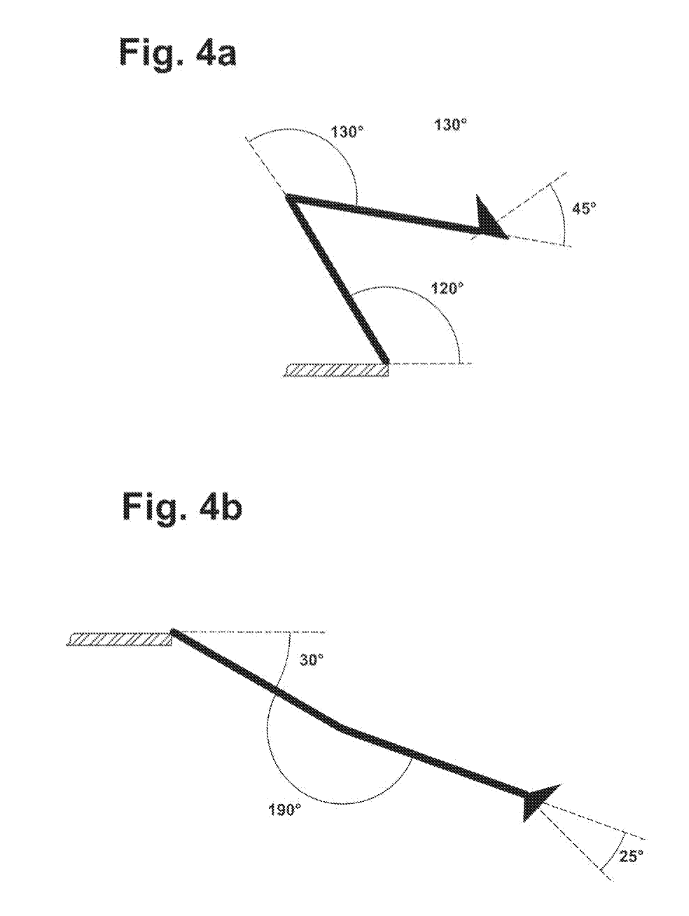

FIGS. 4a and 4b schematically show the two extreme positions of the functional orthosis of the device of FIG. 1 in flexion 4a and in extension 4b;

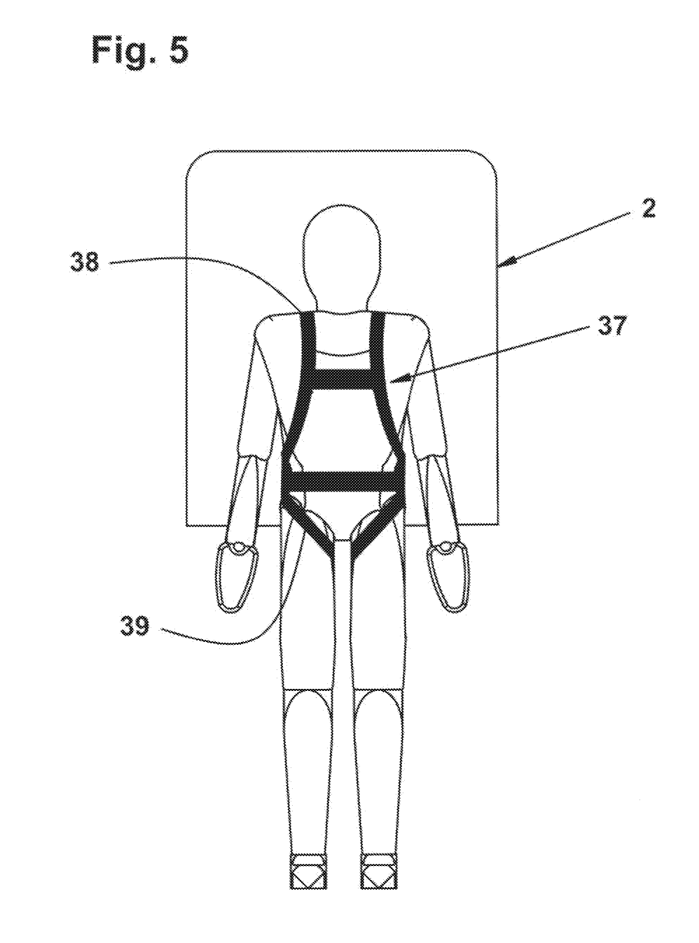

FIG. 5 shows the parachutist-type harness intended to support and hold the patient on the device, in particular in the positions of FIGS. 2 and 3;

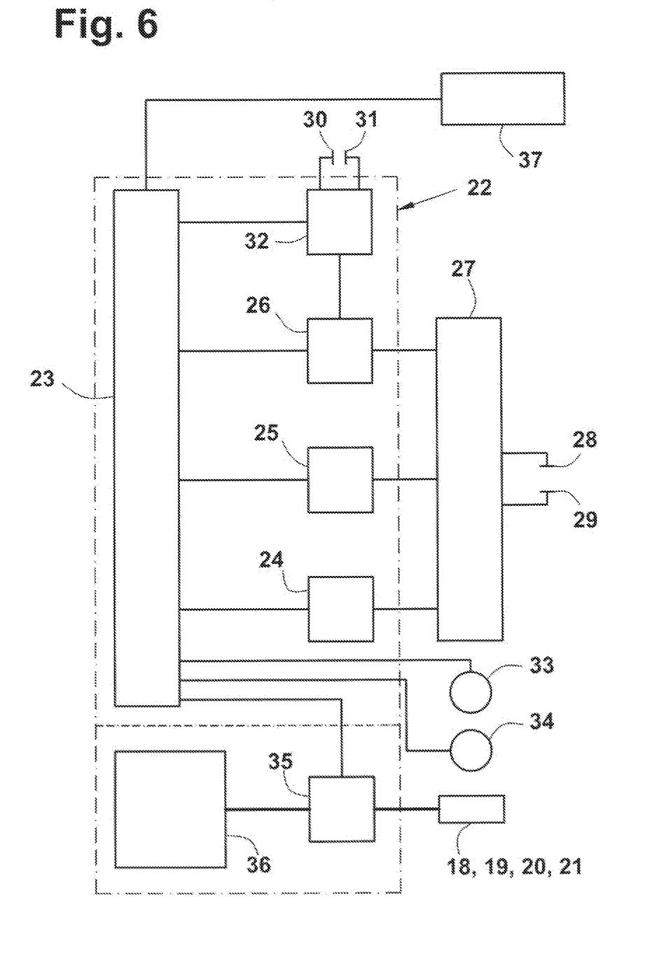

FIG. 6 shows a block diagram of all of the system according to the present invention.

According to the exemplary embodiment of the device represented in FIG. 1, the device comprises a table 2 arranged and articulated on a scissor-based mobile elevator mechanism 3, such that said table, initially horizontal, can be gradually raised and tilted, then indexed in any intermediate position, as illustrated in FIG. 2, to a final vertical position illustrated in FIG. 3. The scissor-based mobile elevator mechanism 3 is itself arranged on a base frame 4 provided with castors 5. At the articulated end of the table 2 there are fixed, via an articulation 6, and by means of a height-adjustable support 7, two identical functional orthoses of exoskeleton type, in other words a functional orthosis for each leg of the patient 1. The table 2, duly padded, is intended to support the back and the pelvis of the patient in initial dorsal decubitus position. Each of the two orthoses constitutes a robotic system of serial type, made up of three segments 8, 9, 10, linked by articulations 11, 12.

Each of the orthoses is arranged so as to produce an exoskeleton for supporting and guiding the lower limb, thus ensuring a mechanical interface with the three bodily segments which make up the lower limb, namely the thigh, the leg and the foot.

To this end, and referring to FIG. 2, the segments of the lower limb, thigh and leg, can be linked to the corresponding segments 8, 9 of the mechanical orthosis by means of trough-shaped padded supports 13, 14 and “Velcro” type closure straps 15, 16 linked to the orthotic structure.

The orthotic segments of the thigh 8 and of the leg 9 are made up of telescopic tubes, the length of which can be adapted to the morphology of the patient, in such a way that the orthotic articulations of the hip 6, of the knee 11 and of the ankle 12 coincide from a functional point of view with the corresponding physiological articulations of the patient. The third orthotic segment 10 constitutes the foot support. The foot is kept constantly pressed against this foot support by means of a flexible structure, which is like the upper structure of a shoe, capable of being closed securely by flexible tongues 17 with “Velcro” type closure.

The anatomy and the biomechanics of the human limbs show an articular and muscular structure of serial type. Thus, the interface described, closely linking the bodily segments of the lower limb to the corresponding orthotic segments of serial type, constitutes a functional unit: the movements of the limb and of the orthosis will consequently be linked and identical.

The robotic structure of serial type of the orthosis consequently allows it to cooperate ideally with the specific serial structure of the segments of the limb and ensure the closest possible mimicry on a physiological level, making it possible to perform with the same simplicity and effectiveness, both the closed muscular chain and articular drivings, such as, for example, the “leg press”, pedaling and the process of gait, and the specific drivings with all the requisite open chain precision of a given articulation and of the muscles involved.

However, the orthoses can also be produced with kinematics of parallel type without departing from the scope of the present invention.

The kinematics of serial type of each orthosis, which comprises only a single kinematic chain, is the simplest. The advantages of such a serial system are fairly numerous, because this system can very easily be adjusted to the morphology of the patient. It can be folded down easily and automatically. Since the three articulations are independent, control is very simple.

But above all, as presented in FIGS. 4a and 4b, the articular mobility allowed by such a system is maximal and allows for an optimal driving of the articular mobility, over its entire physiological extent. In effect, such driving demands, for each of the articulations, the following amplitudes of movements, respectively of extension and of flexing:
articulation of the hip: −30° to 120°
articulation of the knee: −10 ° to 130°
articulation of the ankle: −25° to 45°

The serial orthoses of exoskeleton type comprising the three hip, knee and ankle articulations are placed on either side of the lower limbs, on the outside. Each articulation is driven by a mechanism of connecting rod-crank type and a motorized transmission. This mechanism makes it possible to actuate a crank via a connecting rod. The latter is, in the tradition of light medical robotics, a threaded rod which slides like a plunger cylinder. The telescopic movement is obtained by a nut whose rotation is controlled by an electric motor, thus transforming the rotation of the motor and of the nut into translational movement applied to the articulations of the orthosis. The worm screw also acts as a reducing gear. This “traditional” solution offers the advantage of a reasonable cost and of an installation ensuring ease of local control.

However, this solution shows a number of drawbacks:

The electric actuators have a low power-to-weight ratio, the latter being the ratio between the power developed by the actuator and the weight thereof. The reduction ratio is fixed and limited.

The speed and the torque delivered depend on the reduction ratio: a low reduction ratio allows for a greater speed but a lesser torque, a high reduction ratio reduces the speed but increases the torque.

The capacity of the electric actuator is also limited by the heating of the motor which must consequently be overdimensioned.

In the particular case of the device according to the invention, the actuators of the articulations of the knee and of the ankle represent critical weights since they are “borne” by the orthosis itself often in overhanging position. Thus, the mass inertia itself becomes all the more critical as the speed of movement of the orthosis increases.

The result of a previous production of such a device with electric yet duly calculated actuators is that the power and the speed of the orthoses of the legs may prove only just compatible to ensure an initial driving of the paralyzed lower limbs. However, the speed and the power available prove insufficient to ensure and control movements of the limbs of the device according to the invention and in particular the acceptance of all of the weight of the patient in the driving of the gait process in standing position.

In the case of the device according to the invention, one objective is to obtain, particularly in the case of the driving of the force by full extension of a leg (“leg press” exercise) against load resistance, a maximum force of 2000 Newtons at a maximum speed of 1 m/s.

Another objective is for the leg orthosis to be able, in the context of the driving of the gait in standing position on the foot support 10 of the orthosis, to support all of the weight of the patient in the stance phase of the gait cycle. Bearing in mind that provision is made for a maximum authorized weight of the patient of 140 kg and that, in stance phase, this weight increases more by the order of 20%, the total weight to be accepted is of the order of 170 kg. Consequently, the targeted maximum force of 2000 Newtons is necessary.

To overcome all the drawbacks described previously in the electric actuators and meet the demands of the present invention, provision is made to equip the device according to the invention with hydraulic actuators. The latter offering the following advantages: a power-to-weight ratio of 5 to 10 times greater than the electric actuators, a very high acceleration capability and a high actuation speed, even at maximum torque.

These features and the great rigidity of the hydraulic actuators allow a direct driving, without reducing gear, of the articulations of the orthosis, well suited to the distributed motricity of the articulations of the orthosis and allowing fluid movements.

Another drawback of the electric actuators is that their operation proves too noisy: when associated with their reducing gear, they emit a disagreeable noise of strident nature, further substantially increased by the number of actuators operating simultaneously within an orthotic robotic device. Such a noise results in a proven nuisance for the user and his or her environment.

On the contrary, a system of hydraulic actuators offers the advantage of almost silent operation which provides great user comfort.

The only slightly noisy elements of a hydraulic system are the pump and the electric servovalves which can be grouped together in a sound-proofed central unit.

Finally, the hydraulic actuators offer the additional advantage, contrary to the electric actuators, of not emitting electromagnetic interference. This advantage is significant in immediate proximity to an integrated electromyogram measurement system (EMG) used in combination with the operation of the actuators, as will be described later.

As represented in FIG. 1, the orthotic articulations of the knee 11 and of the ankle 12 are of the connecting rod-crank type. The connecting rod being the rod of the dual-acting hydraulic cylinders 18 and 19. In this arrangement, the connecting rod has an end making a translational movement associated with a pendulum movement linked to the crank.

The orthotic articulation of the hip requiring a total rotational capacity of 150° cannot be of the connecting rod-crank type. A rotary hydraulic cylinder 20 is used to this end.

Each hydraulic cylinder is provided with a position sensor, each orthotic articulation is provided with an absolute angular position sensor 33, and force and torque sensors 34.

Because of the adoption of a system of hydraulic actuators for the mobility of the orthoses provision is also made to use hydraulic actuators, respectively to actuate, by means of a hydraulic cylinder, the scissor-based mobile elevator mechanism 3 of the table 2 and to gradually actuate, by means of a cylinder 21, the transition from the horizontal position of the table 2 represented in FIG. 1 to the vertical position of said table represented in FIG. 3 and to index it at any intermediate tilted position as represented in FIG. 2.

FIG. 5 illustrates, by way of example, a complete harness (37) for supporting the person on the accommodating table 2. This harness comprises a main pelvic belt, fixed by its dorsal part to said table. This belt is provided with a bottom part of leg strap type (39), intended to support the weight of the patient, particularly in vertical position, and a stabilizing abdominal belt.

Shoulder straps (38) are also attached to the rear upper part of the pelvic belt. The other end of the shoulder straps is attached behind the shoulder of the patient to the accommodating table 2. All of the harness is designed to be perfectly adjustable to the size of a given person.

The harness fulfils three functions: 1) the lateral stabilization of the person, 2) in dorsal decubitus position and in the intermediate partial decubitus positions, the shoulder straps counterbalance the reaction force exerted during the driving exercises, particularly in the case of the “leg press” exercise, and 3) in standing position for the gait exercise, the leg strap supports the weight of the person.

Referring to FIG. 6 which shows, by way of exemplary embodiment, the block diagram of a system according to the present invention described hereinbelow.

A central control unit 22 which contains a microcomputer 23 which is the central unit for programming, processing data and controlling all of the system. This microcomputer is connected to various modules or units described hereinbelow. The microcomputer 23 is connected with at least one electric neuromuscular stimulation module 24.

Each output channel of a stimulation module 24 is connected to a switching station 27, responsible for managing a pair of electrodes 28 and 29.

The microcomputer 23 is also connected with at least one electromyogram (EMG) measurement module 25 whose measurement input channel is connected to the switching station 27.

The microcomputer 23 is also connected directly with the switching station 27.

The microcomputer 23 is also connected with a management and control unit 32 of a pair of reference electrodes 30 and 31 of the EMG system connected to the ground of said system.

As a whole, the modular neuromuscular stimulation and electromyogram measurement system presented hereinabove and applied according to the present invention constitutes a “multichannel functional electrical stimulation (FES) and electromyogram measurement (EMG) system described in detail in the Swiss patent application No. 00262/15, and in the international patent application PCT/162016/050896.

The microcomputer 23 is also connected with at least one management and control unit 26 for the electronic identification and authentication microchips incorporated in the electrodes 28 and 29, and 30 and 31. This electrode identification and authentication system applied according to the present invention is described in detail under the title “Surface transcutaneous electrode with incorporated electronic microchip” in the Swiss patent application No. 00263/15, and in the international patent application PCT/IB2016/050896.

The microcomputer 23 is also connected with the position sensors 33 incorporated in each of the articulations 6, 11 and 12 of the orthoses, and in the hydraulic actuators of the scissor-based elevator mechanism 3 of the table 2 and in the tilt actuator 21 of the table 2.

The microcomputer 23 is also connected with the force and torque sensors 34 incorporated in each of the articulations 6, 11 and 12 of the orthoses, which can be mobilized or braked by the hydraulic cylinders 18, 19 and 20.

The position sensors 33 and the force and torque sensors 34 transmit in real time their information to the microcomputer 23. Said management microcomputer of the central control unit 22 interprets these data, which makes it possible to know in real time the articular angular position, the acceleration and the angular speed of each articulation, as well as the forces and torques which are developed therein, as well as the height of the position of the table 2 above the ground and the angle of tilt of said table, which can vary gradually from the initial horizontal position to the vertical position.

This information thus constitutes comprehensive feedback on the activity under load resistance of each articulation. This technical feedback thus replaces the physiological feedback as is normally transmitted to the central nervous system by the proprioceptive nervous system. In fact, the technical feedback supplied by the orthotic serial structure closely mimics the deficient physiological feedback.

The microcomputer 23 is also connected to each of the electro-hydraulic servovalves of the group 35 which controls the hydraulic flow delivered, by the hydraulic power group 36 comprising a tank and a pump, to each hydraulic actuator (hydraulic cylinder) 18, 19, 20, 21 and to the scissor-based elevator mechanism 3.

The microcomputer 23 is finally connected to a human/machine interface 37 with touchscreen for control of the device by the operator, combined with a “biofeedback” system through the display on said screen placed in front of the exercising person, of various data indicating the performance level achieved or to be achieved, notably to motivate and encourage the person to persevere.

The specialist literature highlights the total importance of a mental involvement which can have a marked beneficial complementary effect which facilitates the current activity.

The execution according to a driving program is controlled by a so-called “compliance” device which stores in the microcomputer 23 various data resulting from the execution of said driving, such as, for example, any execution deviations of each of the parameters initially programmed. This compliance can then be consulted and interpreted by the operator.

Referring to FIG. 1, the device is shown in its initial position with the table accommodating the patient in its lowest position, for example at 55 cm above the ground. In this position, the mechanism for adjusting the height of the support 7 also makes it possible to lower the articulation 6 for fixing the orthoses to the table 2 to retract said orthoses below the level of said table as is shown in FIG. 1.

This low arrangement of the table accommodating the patient with the retracted fixing of the orthoses allows for an easy transfer of said patient from his or her wheelchair to said table.

Then, by means of the scissor-based elevator mechanism 3, the table can be raised to an optimal height for the operator and the height of the adjustable support 7 can be adjusted for the articulation 6 fixing the orthoses to cooperate properly with the articulation of the hip of the patient. For the operator, this intermediate position of the height of the table 2 and of the patient greatly facilitates the anthropometric adjustments, notably the adjustment of the harness (37), of the length of the telescopic orthotic segments of the thigh 8 and of the leg 9, of the supports 13 and 14, and of their closure straps 15 and 16, as well as the placement of the electrodes.

Then, by gradual adjustment, respectively of the height of the table 2 and of its tilt in intermediate position in partial decubitus position, the position of the patient is brought to the optimal driving position of a given exercise, such as, for example, “leg press” or of pedaling, or even any other given exercise. An example of said intermediate position is illustrated in FIG. 2.

The gradual adjustment, respectively of the height of the table 2 and of its tilt makes it possible in final position to bring the table 2 to an appropriate height and into vertical position with the patient standing on the foot supports 10 of the orthoses of the legs, while his or her own weight is supported by the harness according to FIG. 5. In this ultimate position shown in FIG. 3, the patient is ready to begin a driving of the gait process.

The essential aim of the device is to be capable of generating movements of the lower limbs with any type of duly controlled trajectories, executed against programmed fixed or changing load resistances.

The kinematics of the trajectory of a given movement are calculated and stored in the microcomputer 23. These kinematics of a given trajectory can be reproduced with total fidelity because each of the two functional orthoses motorized by the hydraulic actuators 18, 19, 20 constitutes a robotic system of serial type made up of three segments 8, 9, 10 linked by the articulations 11, 12 to the work table 2 by the articulation 6. The position sensors 33 and the force and torque sensors 34, incorporated in each of said articulations of the orthosis, allow for the real time closed loop adjustment of said trajectory.

Initially, a given trajectory is calculated to be reproduced passively, with zero force exerted by the patient, only the specific weights of the orthosis and the weight of the leg are taken into account and neutralized, in particular against gravity. In this case, there are no active interactions of the subject with the orthosis.

Then, the same trajectory is accomplished in a voluntary active manner by a healthy subject and the sequence of electromyograms (EMG) of the muscles involved is duly measured and recorded. This procedure can be repeated with different load resistance values applied to the articulations.

An identical procedure can be carried out, in the standing position of the subject, for the specific trajectory of the legs during a gait cycle.

The trajectory reproduced passively, with zero force exerted by the patient, is performed while all the weight of the patient is supported by his or her harness, which results in a total absence of force applied to the foot support 10 and consequently no reaction force exerted by said foot support.

The same trajectory of the gait cycle is then accomplished in a voluntary active manner by a healthy subject and the sequence of electromyograms (EMG) of the muscles involved is duly measured and recorded. This procedure must be repeated with different values of the force applied to the foot support 10 provoking an equivalent reaction force exerted by said foot support, a force which will correspond to the proportion of weight of the subject freed of its support by the harness.

This variation of the force applied to the foot support 10 can be obtained by a very slight variation of the trajectory of said foot support produced by a very slight modulation of the length of the radius of circle described between said foot support and the orthotic articulation of the hip 6. When said radius of circle is very slightly reduced the force applied to the foot support 10 increases and the reaction force exerted by said foot support increases commensurately, which provokes an equivalent alleviation of force in the supporting harness of the subject.

This slight modulation of said radius of circle can range up to the total application of the weight of the body on the foot support 10 and therefore the complete alleviation at the harness level.

Even in this extreme case, the necessary shortening of said radius of circle does not significantly hamper an adequate cooperation of the orthotic segments with the bodily segments of the leg allowing a correct accomplishment of the gait cycle without restriction.

By this same real time operation of modulation of said radius of circle during a gait cycle, it is possible to modulate the reaction force exerted by the foot support 10 to respect a close mimicry with the modulation of the reaction force of the ground that is well known occurring during a voluntary gait cycle.

When the voluntary execution of the trajectory of a given movement is not possible, because of a paralysis of the muscles involved or is deficient because of a partial paralysis of said muscles, the text of the description of the invention above has stressed the major drawback resulting from a passive driving imposed by a manual or robotic external means, and, by contrast, the complete primordial and physiologically incontrovertible importance of the active participation of the muscles involved.

It is also described that, in such cases, the active participation of the muscles can be effectively provoked by a stimulation of said muscles by means of a functional electrical stimulation of the muscles adjusted in closed loop mode in real time of CLIMFES (Closed-Loop Integrated Myography Functional Electrical Stimulation) type.

To produce such a stimulation, there is normally a complex preliminary modeling of each given muscle which includes the stimulation intensity (mA). The model also takes into consideration the influence of the articular angles. This is modeled by two cubic functions, one for each articulation in the case of bi-articular muscles. The identification is accomplished at various determined angles and intensities. The total identification time for the force intensity and force position relationships is consequently approximately 10 minutes. The final result of this type of modeling is that the adapted regulator is complex. Such a modeling has been described in particular in the patent U.S. Pat. No. 7,381,192.

The complexity of the modeling and of the regulator, the number and the complexity of the computations which have to be executed slowing down the process, and above all the identification time of 10 minutes necessary before each driving session has many drawbacks.

According to the present invention, a different stimulation strategy to overcome the abovementioned drawbacks is applied.

The device comprises an integrated system for the prior measurement and recording of the sequence of the electromyograms (EMG) of all the muscles involved in the execution of a defined trajectory, this sequence defines, throughout said trajectory and for each muscle, a stimulation window as a function of the position of its EMG, which also prevents said muscle from being stimulated at non-physiological moments. The force position relationship also ensues from said stimulation window. The force intensity relationship is also based on the EMG measurements.

For a predefined trajectory, executed in a voluntary active manner by a healthy subject without any assistance from the device of the invention and by means of his or her own physical capabilities, all the reference data of the corresponding sequence of the EMGs measured can be recorded online in a look-up table incorporated in said system.

Moreover, during the execution of said predefined trajectory, the measurements performed by the articular position sensors 33 and/or the force sensors 34 can be recorded in a look-up table incorporated in the system that can cooperate with the look-up table of the EMGs.

Such an implementation has the advantage of being simple, precise and very rapid. Because the look-up table represents precisely the desired trajectory, no approximation is made.

The result thereof is that the EMG measurements provide the correct information for synchronization of the electrical stimulation. While the force sensors 34 allow the adjustment of the intensity of the electrical stimulation. The combination of the EMG measurements and of said force sensors 34 finally allows for a neuromuscular stimulation that is adjusted in closed loop mode in real time of the CLIMFES (Closed-Loop Integrated Myography Functional Electrical Stimulation) type.

Consequently, a simple regulator intended to adjust the hydraulic actuators 18, 19 and 20 can be of conventional PID (proportional integral derivative) type. There is no longer any need for more complex implementation to control the orthoses of the legs.

The device described above offers an extreme application versatility, because it is capable of generating and controlling movements of the lower limbs executing any type of duly controlled physiological trajectories, executed against programmed fixed or changing load resistances. It also enables both closed chain muscular and articular drivings and open chain specific drivings of a given articulation.

It allows, in dorsal or partial decubitus position, the driving of the mobility, of the force and of the endurance of the lower limbs and, in vertical position, the early driving of the gait.

Through its integrated system combining a stimulation of CLIMFES type with the EMG measurements, it ensures a physiologically optimal driving with an active participation of the muscles which, associated with the respect for the closest mimicry of the voluntary activity that has become impossible or restricted following an injury to the central nervous system, guarantees a correct proprioceptive stimulation of the central nervous system and, if necessary, of its plasticity of substitution and of objectively assessing the early signs of a functional recovery, then its evolution in time by means of the integrated surface electromyography measurement system (EMG).

Finally, it allows for an overall optimal driving of a person in motor or neuromotor handicap situation, such as, for example, paraplegia, hemiplegia, and in case of cerebral palsy; and in the field of sport for functional post-traumatic re-education and/or driving.

Following the description which has just been given, in order to illustrate how the invention can be produced advantageously, it should be noted that the invention is not limited to this embodiment. Several variant embodiments of a device for driving the lower limbs and upper limbs of a person in dorsal or partial decubitus position combined with the driving of the gait in vertical position can be envisaged in the field of those skilled in the art without departing from the scope of the present invention as defined in the attached claims.

1. A device for driving the lower limbs of a person, comprising a base frame, a table supporting the person, at least one motorized mechanical orthosis arranged to constitute an interface with at least one of the lower limbs of said person such that the movements of said lower limb and of said orthosis are linked and identical, said orthosis being fixed to an end of said table, and a functional electrical stimulation and electromyogram measurement device comprising at least one pair of stimulation and measurement electrodes intended to act on a muscle or muscle group of said lower limb to, on the one hand, stimulate said muscle or muscle group, and, on the other hand, measure the reaction of said muscle or muscle group, said device further comprising an elevation mechanism making it possible to vary a vertical position of the table relative to the base frame between a low position, in which the transfer and the installation of the person are facilitated, intermediate working positions, and a high position allowing a driving of the person in standing position and a mechanism for tilting said table making it possible to vary the tilt of said table relative to the base frame, in particular between a horizontal position, in which the person is in dorsal decubitus position, and a vertical position, in which the person is in standing position, the combination of the elevation and tilt mechanisms of the table allowing the mobility of said orthosis over the entire extent of the respective physiological ranges of movements of said lower limb.

2. The device as claimed in claim 1, further comprising two orthoses, each being arranged to constitute an interface with one of the lower limbs of the patient.

3. The device as claimed in claim 1, wherein each orthosis comprises at least three orthotic segments intended to constitute a mechanical interface with, respectively, a thigh, a leg and a foot of the person, the first and second segments comprising means for their respective links to the thigh and to the leg of the person and the third segment being arranged so as to constitute a foot support and comprising means for fixing same to the foot of the person, the first segment being linked, at one of its ends, to the table by a first motorized articulation positioned at the level of the hips of the person, and, at its other end, to one of the ends of the second segment by a second motorized articulation, the other end of said second segment being linked by a third motorized articulation to the third segment, the motorization of the articulations being ensured by actuators.

4. The device as claimed in claim 3, wherein the link means of the first and second orthotic segments consists of trough-shaped padded making it possible to accommodate one of the lower limbs of the person and of straps linked to said orthotic segments.

5. The device as claimed in claim 3, wherein the third orthotic segment has the general form of a shoe making it possible to accommodate one of the feet of the person, said shoe being securely closed by means of flexible tongues.

6. The device as claimed in claim 3, wherein the actuators are of hydraulic type, in particular dual-acting hydraulic cylinders for the second and third articulations and a rotary hydraulic cylinder for the first articulation.

7. The device as claimed in claim 3, wherein the first and second orthotic segments consist of elements of variable lengths, so as to be able to adapt their length to the morphology of the person.

8. The device as claimed in claim 3, wherein the first articulation is fixed adjustably to the table such that it can be retracted below the level of the table in horizontal position to facilitate the transfer and the installation of the person on said table.

9. The device as claimed in claim 3, wherein each articulation is provided with at least one position sensor and at least one force sensor, said sensors being capable of transmitting in real time, to a central control unit, data relating to the position of said articulation and to the forces and torques which develop therein.

10. The device as claimed in claim 9, wherein the central control unit contains at least one reference data table, in which have been stored a series of electromyogram measurements performed by the pair of stimulation and measurement electrodes in relation to a series of measurements performed by the position and force sensors, said reference data relating to a reference user having mobilized each of his or her lower limbs according to a predefined trajectory by means of just his or her physical capabilities, said central control unit using said reference data table to control the actuators and the pair of stimulation and measurement electrodes in closed loop mode and in real time so as to mobilize at least one of the lower limbs of the person according to said predefined trajectory.

11. The device as claimed in claim 9, wherein the central control unit comprises a microcomputer.

12. The device as claimed in claim 1, wherein the tilt mechanism of the table comprises at least one actuator.

13. The device as claimed in claim 1, wherein the table supporting the person is equipped with a harness supporting said person.

14. The device as claimed in claim 13, wherein the harness comprises an upper part with shoulder straps intended, in horizontal or tilted position of the table with the person in dorsal or partial decubitus position, to counterbalance the reaction force exerted during force driving exercises, and a lower part of leg strap type, intended, in vertical position, to support the weight of said person.

15. The device as claimed in claim 1, wherein the elevation mechanism comprises a scissor structure incorporated in the base frame.
